(19) United States
(12) Patent Application Publication
(10) Pub. No.: US 2026/0144436 A1
(43) Pub. Date: May 28, 2026

(54) METHOD FOR CLEANING SURGICAL INSTRUMENTS

(71) Applicant: OLYMPUS Winter & Ibe GmbH, Hamburg (DE)

(72) Inventor: Sascha ESCHBORN, Ahrensburg (DE)

(73) Assignee: OLYMPUS Winter & Ibe GmbH, Hamburg (DE)

(21) Appl. No.: 19/453,204

(22) Filed: Jan. 20, 2026

Related U.S. Application Data
(63) Continuation-in-part of application No. 18/138,909, filed on Apr. 25, 2023, now Pat. No. 12,551,316.
(60) Provisional application No. 63/334,743, filed on Apr. 26, 2022.

(51) Int. Cl. A61B 1/12; A61B 50/13

(52) U.S. Cl. CPC A61B 1/123; A61B 1/125; A61B 50/13

(57) ABSTRACT

A method for cleaning a surgical instrument, the method including pre-cleaning the surgical instrument in a transport trolley with a pre-cleaning fluid while the surgical instrument is being transported to a processing device for cleaning the surgical instrument; and subsequent to the pre-cleaning, cleaning the surgical instrument using the processing device.

### CROSS-REFERENCE TO RELATED APPLICATION

The present application is a Continuation-In-Part Application of U.S. patent application Ser. No. 18/138,909, filed on Apr. 25, 2023, which is based upon and claims the benefit of priority from U.S. Provisional Application No. 63/334,743 filed on Apr. 26, 2022, the entire contents of each of which are incorporated herein by reference.

### BACKGROUND

### Field

The present disclosure relates to a method for cleaning a surgical instrument, such as an endoscope.

### Prior Art

It is well known in the prior art that transport trolleys are used for the transport of endoscopes between an operation room and the room for reprocessing the endoscopes. After a surgery or a medical examination, the endoscopes are cleaned manually first and then disinfected or reprocessed automatically. In order to prevent residual dirt from drying in the endoscope channels and thus leading to blocked channels, the time between intervention and manual cleaning, as well as automated process, is limited. The manual cleaning of the surgical instruments is to be carried out typically within two hours. Moreover, the time between the manual cleaning and the automatic reprocessing of the surgical instruments shall not exceed approximately 30 minutes.

These time requirements for the manual cleaning and automatic reprocessing of surgical instruments entails personnel which must always be available to perform transport as well as manual and mechanical cleaning steps of the surgical instruments, such as when surgical instruments like endoscopes are used for surgeries at nights or on weekends.

### SUMMARY

An object is to simplify or to improve the cleaning processes of surgical instruments significantly.

Such object can also be solved by a method for cleaning a surgical instrument, such as an endoscope, using a transport trolley, wherein a pre-cleaning of the surgical instrument with a pre-cleaning fluid, which can be automatic, of at least one or more channels of the surgical instrument can be carried out.

Furthermore, after the pre-cleaning of the surgical instrument by the pre-cleaning device of the transport trolley, the surgical instrument can be processed by processing device, which can be stationary.

The method for cleaning a surgical instrument, can comprise pre-cleaning the surgical instrument in a transport trolley with a pre-cleaning fluid while the surgical instrument is being transported to a processing device for cleaning the surgical instrument; and subsequent to the pre-cleaning, cleaning the surgical instrument using the processing device.

The cleaning can be performed in the processing device while the processing device can be stationary.

Moreover, the pre-cleaning can comprise one or more of sending first pre-cleaning fluid through one or more internal channels of the surgical instrument and applying second pre-cleaning fluid to an outside of the surgical instrument.

In one embodiment, the first pre-cleaning fluid can be the same as the second pre-cleaning fluid.

According to another embodiment, the pre-cleaning can be automatically carried out on the transport trolley.

For example, the pre-cleaning can be performed in at least one pulsation mode of the pre-cleaning fluid. By the pulsation of the pre-cleaning fluid, which can be a pre-cleaning liquid, the removal of dirt or microorganisms or bacteria or other deposits, like biofilms or protein residues, the pre-cleaning process can be improved. The pre-cleaning process comprises at least one of the step of pre-cleaning the channels inside of the surgical instrument and of the step of pre-cleaning the outer surfaces or the outside of the surgical instrument.

Furthermore, the at least one pulsation mode of the pre-cleaning fluid can comprise one or more of varying the pressure of the pre-cleaning fluid and of varying the flux rate of the pre-cleaning fluid and of varying the duration of the pre-cleaning. The pre-cleaning process can be carried out for example by pulsing the pressure of the pre-cleaning fluid, which for example is pumped by a fluid pump through a fluid feed line by varying the pressure of the pre-cleaning fluid or by varying the flux rate of the pre-cleaning fluid. In addition, the durations or the frequencies of the pre-cleaning steps can be also varied. Within the scope of the invention, also other pulsation parameters of the pre-cleaning fluid can be varied.

The at least one pulsation mode of the pre-cleaning fluid can be controlled depending on one or more of providing detected data of the pressure of the pre-cleaning fluid and of providing detected data of the flux rate of the pre-cleaning fluid and of providing detected data of an optical measurement of the pre-cleaning fluid. For the control of the pulsation mode or pulsation modes, a sensor for detecting the pressure of the pre-cleaning fluid, for example in the feed line, and/or a sensor for detecting the flux rate of the pre-cleaning fluid can be provided. Moreover, the transport trolley can also comprise a sensor for detecting or for monitoring the optical properties, like the turbidity, of the pre-cleaning fluid.

In one further embodiment, the at least one pulsation mode of the pre-cleaning fluid can be adaptively controlled during the pre-cleaning. The at least one pulsation mode for the pre-cleaning process can be controlled by a controller or the like, wherein for example the controller carries out a software-based and/or adaptive algorithm or an AI based algorithm, like a self-learning and/or adaptive algorithm, for the pre-cleaning process.

In addition, the transport trolley can be provided at least with one or more of a sensor for detecting the pressure of the pre-cleaning fluid, a sensor for detecting the flux rate of the pre-cleaning fluid and a sensor for detecting at least one optical property of the pre-cleaning fluid.

The mobile transport trolley for surgical instruments, such as endoscopes, can have at least one receiving compartment for receiving an instrument tray, such as a closable instrument tray, for arranging a surgical instrument in the instrument tray, and a pre-cleaning device for a surgical instrument to be pre-cleaned which is arranged or can be arranged in the instrument tray, wherein the pre-cleaning device is connectable or connected to the surgical instrument to be pre-cleaned by a coupling device and/or having a pre-cleaning apparatus for pre-cleaning an outside of the surgical instrument.

The transport trolley can have at least one receiving compartment or several compartments for receiving an instrument tray with a surgical instrument positioned therein, wherein the surgical instrument in the instrument tray or in each instrument tray is pre-cleaned by a pre-cleaning device of the transport trolley, wherein the pre-cleaning device is connectable or connected with the surgical instrument in the instrument tray. After positioning the instrument tray with the surgical instrument in the corresponding receiving compartment and after a connection the pre-cleaning device with the surgical instrument, a pre-cleaning process of the surgical instrument, which can be automatic, can be carried out on the transport trolley.

The transport trolley can comprise several, for example three to six or more, receiving compartments for receiving an instrument tray with a surgical instrument. The surgical instrument itself can comprise at least one or more channels, which has or have to be pre-cleaned on the transport trolley. After positioning the instrument tray in the receiving compartment, the channels of the surgical instrument, such as an endoscope, can be connected or coupled with the pre-cleaning device by f one or more coupling devices, such as adapters. Further pre-cleaning process of the surgical instrument will be or is performed by activating the pre-cleaning device on the transport trolley. Therefore, the instrument tray can have a cover or the like in order to close the instrument tray for the pre-cleaning process.

In addition or as an alternative, the outside or the outer surfaces of the surgical instrument can be pre-cleaned by a pre-cleaning apparatus. Therefore, the external control knobs, control buttons or control wheels of the surgical instruments or the outside of the endoscope shaft or the like are pre-cleaned, wherein the surgical instrument is arranged in the instrument tray, which can be closed.

In addition, the transport trolley can be movable and comprises for example wheels or the like.

In order to perform the pre-cleaning of the surgical instrument in the instrument tray on the transport trolley, the pre-cleaning device can comprise a fluid supply for a pre-cleaning fluid for the surgical instrument and/or the pre-cleaning apparatus can comprise a fluid supply for a pre-cleaning fluid for the surgical instrument. The pre-cleaning fluid can be a pre-cleaning liquid and/or a pre-cleaning gel or a pre-cleaning gel-like mass or the like. By pumping the pre-cleaning liquid through one or more channels of the surgical instrument, the contamination of bacteria, dirt, such as visible dirt, or the like are reduced significantly.

The coupling device can be configured such that at least one channel or several channels of the surgical instrument is or are fluidically connected to the fluid supply for the pre-cleaning fluid. For example, by using the pre-cleaning fluid or liquid, the visible dirt as well as bacterial contamination can be reduced in such a way that the steps of a manual cleaning, such as on the transport trolley, before an automatic cleaning and disinfection process by a reprocessing device in a reprocessing room can be skipped. In addition, the pre-cleaned surgical instruments, such as endoscopes, can be stored on the transport trolley for a long time, which can lead to a considerable reduction in personnel for the reprocessing of surgical instruments.

The pre-cleaning device can comprise a fluid pump connected to the fluid supply by a fluid feed line. After connecting the pre-cleaning device with one or more channels of the surgical instrument via the coupling device, such as an adapter, the fluid pump pumps the pre-cleaning fluid from the fluid supply into the channels of the surgical instrument in order to remove the dirt or microorganisms or bacteria inside the channels for the pre-cleaning process on the transport trolley.

In order to improve the pre-cleaning process on the transport trolley, a fluid return line can be provided for the pre-cleaning fluid emerging out or leaking from the surgical instrument. The fluid return line can be connected by a coupling device with the outlet of the channels of the surgical instrument. After flowing through the channels of the surgical instrument, the pre-cleaning fluid emerges out at the outlet side of the channels by flowing into the fluid return line and by discharging by the fluid return line.

In one embodiment of the transport trolley, the fluid return line can be connected to a fluid container for the pre-cleaning fluid and/or a filter for the pre-cleaning fluid is arranged in the fluid return line and/or a pump is arranged in the fluid return line. Thereby, the used discharging pre-cleaning fluid is collected in the fluid container, wherein it is possible that the returning pre-cleaning fluid can be filtered. In addition, a pump can be arranged in the fluid return line, in order to pump the pre-cleaning fluid into the fluid container on the transport trolley. The pre-cleaning fluid or solution emerging e.g. out of the channels of the surgical instrument of from a distal end of the surgical instrument can be collected and can be recycled by a filter system in a further embodiment so that recycled fluid can be (re)used for flushing or pre-cleaning the surgical instruments for a longer term.

The fluid return line can be configured in such a way that the fluid return line can be coupled or is coupled to at least one channel or several channels of the surgical instrument, such as by an adapter.

Moreover, the fluid container can be fluidically connected to the fluid supply by a fluid line, wherein a filter for the pre-cleaning fluid and/or a pump for the pre-cleaning fluid can be provided in the fluid line.

The transport trolley can be provided with an identification sensor for detecting an identification code provided on the surgical instrument, such as the instrument to be pre-cleaned. By the identification sensor, the surgical instrument to be cleaned or pre-cleaned in the tray can be easily identified and recorded for the whole cleaning process. Thereby, it is recorded which surgical endoscope is positioned in the instrument tray.

The identification sensor can be configured as a scanner or as a barcode scanner or as an RFID sensor (radio frequency identification sensor). In this context, the instrument tray and/or the surgical instrument can be provided with an identification tag or the like or with a barcode or a RFID tag.

It is also provided in one embodiment of the transport trolley that the pre-cleaning device can comprise a controller for controlling at least one pre-cleaning process for pre-cleaning the surgical instrument on the transport trolley. The controller is configured to enable a pre-cleaning process, which can be automatic, of the surgical instruments on the transport trolley before the reprocessing process by a cleaning and disinfection reprocessing device in a reprocessing room or area.

Furthermore, the pre-cleaning device can be configured to pre-clean at least one channel or several channels of the surgical instrument by the pre-cleaning fluid, such as in a pre-cleaning process.

The pre-cleaning apparatus can comprise a nozzle and/or an outlet port, wherein the nozzle and/or the outlet port are arranged in the instrument tray. Thereby, the outer surface or the outside as well as the outer control knobs, control buttons or control wheels are pre-cleaned by the pre-cleaning fluid in contact with the outside of the instrument.

The pre-cleaning apparatus can comprise a fluid supply for a pre-cleaning fluid for pre-cleaning the outside of the surgical instrument. The fluid supply for the pre-cleaning apparatus can be different from the fluid supply for the pre-cleaning device. In one embodiment, the pre-cleaning fluid can be provided in one common fluid supply for the pre-cleaning apparatus and the pre-cleaning device.

The pre-cleaning apparatus can comprise a fluid pump connected to the fluid supply by a fluid feed line. In this context, the pre-cleaning apparatus and the pre-cleaning device can have the same fluid pump or separate fluid pumps.

A fluid outlet line can be provided for the pre-cleaning fluid emerging out or leaking from the instrument tray. Thereby, the pre-cleaning fluid for the pre-cleaning of the outside of the surgical instruments can flow or can be discharged out of the instrument tray, which can be closed, after pre-cleaning the outside of the surgical instrument.

The fluid outlet line can be connected to a fluid container for the pre-cleaning fluid and/or a filter for the pre-cleaning fluid can be arranged in the fluid outlet line and/or a pump can be arranged in the fluid outlet line.

In addition, such object can be solved by a surgical system comprising a transport trolley as described above. In this context, reference is expressly made according to the embodiments as described above.

### BRIEF DESCRIPTION OF THE DRAWINGS

Further features will become evident from the description of embodiments, together with the claims and the appended drawings. Embodiments can fulfill individual features or a combination of several features.

The embodiments are described below, without restricting the general idea of the invention, based on the exemplary embodiments in reference to the drawings, whereby we expressly refer to the drawings with regard to the disclosure of all details that are not explained in greater detail in the text. In the drawings:

FIG. 1 illustrates a schematic cross-sectional representation of a transport trolley for surgical instruments;

FIG. 2a illustrates a schematic cross-sectional representation of a transport trolley for surgical instruments according to another embodiment.

FIG. 2b illustrates a schematic cross-sectional representation of a transport trolley for surgical instruments according to another embodiment.

FIG. 3 illustrates a schematic cross-sectional representation of a transport trolley for surgical instruments according to another embodiment.

### DETAILED DESCRIPTION

In the drawings, the same or similar elements and/or parts are, in each case, provided with the same reference numerals such that they are not introduced again in each case.

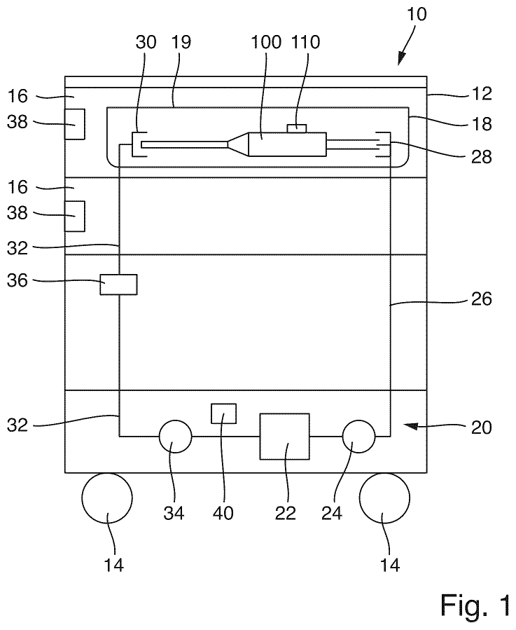

In FIG. 1 shows, in a schematic cross-section view, a mobile transport trolley 10 for surgical instruments 100, such as endoscopes. The mobile transport trolley 10 comprises a rack 12 with wheels 14 at the bottom. The rack 12 is provided with receiving compartments 16 for receiving an instrument tray 18 (one of which is illustrated in FIG. 1). In the instrument tray 18, one surgical instrument 100 is arranged after the use of the surgical instrument, for example for a surgery.

The instrument tray 18 comprises a pivotable, cover 19, which can be pivotable, in order to close the instrument tray after accommodating the surgical instrument 100 inside the instrument tray 18. The closed instrument tray 18 is positioned in the receiving compartment 16 for pre-cleaning the surgical instrument on the transport trolley 10.

Moreover, the transport trolley 10 comprises a pre-cleaning device 20 for the pre-cleaning process of the surgical instrument 100. The pre-cleaning device 20 has a fluid supply 22, which is filled with a pre-cleaning fluid or liquid for the surgical instrument 100. The fluid supply 22 is connected with the surgical instrument 100 via a pump 24, wherein the pump 24 is connected with the surgical instrument by a fluid feed line 26. At the end of the fluid feed line 26 an adapter 28 or connector is provided in order to connect one or more channels (hereinafter channels) of the surgical instrument to the fluid feed line 26.

The pump 24 pumps the pre-cleaning fluid from the fluid supply 22 into the channels of the surgical instrument 100. The pre-cleaning fluid emerges out of the channels of the surgical instrument 100 at the outlet side of the channels. At the outlet side of the channels a connector 30 is provided in order to connect the outlet side of the channels of the surgical instrument with a fluid return line 32. The emerging pre-cleaning fluid flows or is discharged through the fluid return line 32 and is pumped by a pump 34 in the fluid return line 32 into the fluid supply 22. In the fluid return line 32 a filter 36 is provided in order to filter the unclean returned or discharged pre-cleaning fluid.

Furthermore, the rack 12 of the transport trolley 10 has an identification sensor 38 or the like corresponding to each receiving compartment 16 in order to identify the surgical instrument 100, which has to be pre-cleaned on the transport trolley 10. Therefore, the surgical instruments 100 are provided with an RFID tag 110. The information provided by the RFID tag can be read by the identification sensor 38.

In addition, the transport trolley 10 comprises a controller 40 configured to control the pre-cleaning processes of the surgical instrument 100 by the pre-cleaning device 20 arranged on the transport trolley 10. The controller can be hardware or any combination of hardware and software and also have a corresponding memory.

In a further (non-shown) embodiment, the fluid return line 32 can be connected with a separated fluid container in order to collect the pre-cleaning fluid emerging out of the channels of the surgical instrument 100.

The transport trolley 100 is used for a pre-cleaning process of the surgical instruments 100, wherein the used surgical instruments are accommodated in the receiving compartments 16 of the transport trolley 10 for pre-cleaning the surgical instruments, before the pre-cleaned surgical instruments are transported or moved to a reprocessing and disinfection device, which can be stationary.

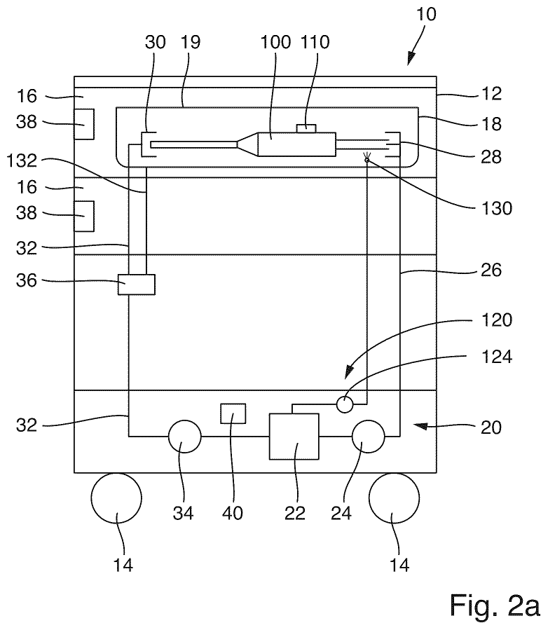

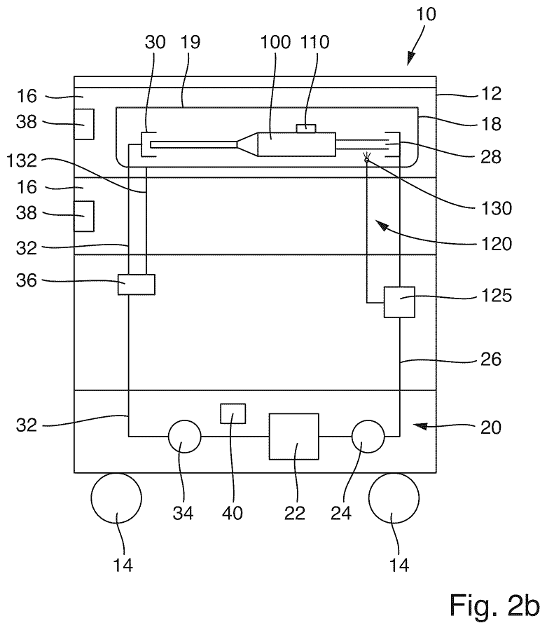

The embodiments shown in FIG. 2a and FIG. 2b illustrate each schematically of a transport trolley for surgical instruments in cross-section. In comparison to the embodiment of FIG. 1, the transport trolley 10 in FIG. 2a comprises a pre-cleaning apparatus 120 for the pre-cleaning of the outside of the surgical instrument 100, wherein a pump 124 is connected with the fluid supply 22 in order to pump or to convey a pre-cleaning fluid to a nozzle 130, which sprays the pre-cleaning fluid onto the outer surface of the surgical instrument 100 in the instrument tray 18. Hereby, the outer surface as well as outer operating units, such as control knobs, buttons and/or wheels etc., of the surgical instrument 100 are pre-cleaned by the pre-cleaning fluid. The pre-cleaning fluid for the outer surface of the surgical instrument is discharged out of the instrument tray by the fluid outlet line 132.

According to the embodiment of FIG. 2b, the fluid supply 22 is connected via one common pump 24 and a valve 125 with the adapter 28. Moreover, the valve 125 in the feed line 26 is also connected with the nozzle 130 in order to pre-clean the outside of the surgical instruments 100. Depending on the switching or the position of the valve 125, the inner channels of the surgical instrument 100 and/or the outside of the surgical instrument 100 is pre-cleaned by the pre-cleaning fluid.

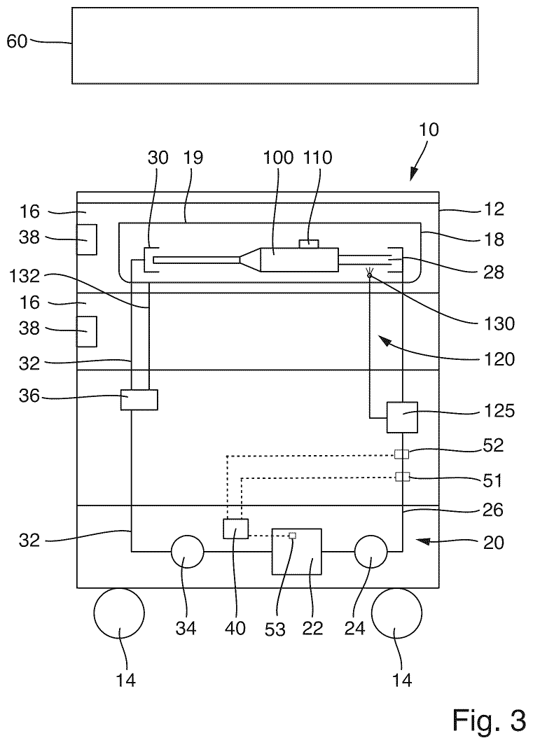

The embodiment shown in FIG. 3 illustrates schematically of a transport trolley for surgical instruments in cross-section. In comparison to the embodiment of FIG. 2b, the transport trolley 10 in FIG. 3 comprises a sensor 51 for monitoring the pressure of the pre-cleaning fluid in the fluid feed line 26. Moreover, a sensor 52 is provided in the fluid feed line 26 in order to monitor the flux rate of the pre-cleaning fluid. In addition, a sensor 53 is provided in the fluid supply 22 in order to monitor the optical properties of the pre-cleaning fluid, like the turbidity of the pre-cleaning fluid. The sensors 51, 52 and 53 are connected with the control unit 40, wherein the control unit 40 is configured to carry out a pulsation mode for the pre-cleaning process of the surgical instrument 100. After the pre-cleaning of the surgical instrument 100 on the transport trolley 10, the surgical instrument 100 is transported to a stationary processing device 60 for the reprocessing of the surgical instrument 100.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

10 Transport trolley
12 Rack
14 Wheel
16 Receiving compartment
18 Instrument tray
19 Cover
20 Pre-cleaning device
22 Fluid supply
24 Pump
26 Fluid feed line
28 Adapter
30 Connector
32 Fluid return line
34 Pump
36 Filter
38 Identification sensor
40 Control unit
51 Sensor
52 Sensor
53 Sensor
60 Processing device
100 Surgical instrument
110 RFID tag
120 Pre-cleaning device
124 Pump
125 Valve
130 Nozzle
132 Fluid outlet line

What is claimed is:

1. A method for cleaning a surgical instrument, the method comprising:
pre-cleaning the surgical instrument in a transport trolley with a pre-cleaning fluid while the surgical instrument is being transported to a processing device for cleaning the surgical instrument; and
subsequent to the pre-cleaning, cleaning the surgical instrument using the processing device.

2. The method according to claim 1, wherein the cleaning is performed in the processing device while the processing device is stationary.

3. The method according to claim 1, wherein the pre-cleaning comprising one or more of sending first pre-cleaning fluid through one or more internal channels of the surgical instrument and applying second pre-cleaning fluid to an outside of the surgical instrument.

4. The method according to claim 1, wherein the first pre-cleaning fluid is the same as the second pre-cleaning fluid.

5. The method according to claim 1, wherein the pre-cleaning is automatically carried out on the transport trolley.

6. The method according to claim 1, wherein the pre-cleaning is performed in at least one pulsation mode of the pre-cleaning fluid.

7. The method according to claim 6, wherein the at least one pulsation mode of the pre-cleaning fluid comprises one or more of varying a pressure of the pre-cleaning fluid, varying a flux rate of the pre-cleaning fluid and varying a duration of the pre-cleaning.

8. The method according to claim 6, wherein the at least one pulsation mode of the pre-cleaning fluid is controlled depending on one or more of providing detected data of the pressure of the pre-cleaning fluid, providing detected data of the flux rate of the pre-cleaning fluid and providing detected data of an optical measurement of the pre-cleaning fluid.

9. The method according to claim 6, wherein the at least one pulsation mode of the pre-cleaning fluid is adaptively controlled during the pre-cleaning.

10. The method according to claim 8, wherein the transport trolley is provided with one or more of a sensor for detecting the pressure of the pre-cleaning fluid, a sensor for detecting the flux rate of the pre-cleaning fluid and a sensor for detecting at least one optical property of the pre-cleaning fluid.

11. The method according to claim 1, wherein the transport trolley comprises:
at least one receiving compartment configured to receive an instrument tray; and
one or more of:
a pre-cleaning device configured to pre-clean a surgical instrument in the instrument tray, the pre-cleaning device is configured to be connected to the surgical instrument by a coupling; and
a pre-cleaning apparatus configured to pre-clean an outside of the surgical instrument.

12. The method according to claim 11, further comprising a fluid supply configured to hold the pre-cleaning fluid for use with one or more of the pre-cleaning device and the pre-cleaning apparatus.

13. The method according to claim 12, further comprising a coupling device configured to fluidly connect one or more channels of the surgical instrument to the pre-cleaning fluid in the fluid supply.

14. The method according to claim 12, further comprising a fluid pump connected to the fluid supply by a fluid feed line.

15. The method according to claim 12, further comprising a fluid return line for removing the pre-cleaning fluid from the instrument tray.

16. The method according to claim 11, further comprising a controller configured to control at least one pre-cleaning process for pre-cleaning the surgical instrument.

17. The method according to claim 11, wherein the pre-cleaning device is configured to pre-clean one or more channels of the surgical instrument by pre-cleaning fluid.

18. The method according to claim 11, wherein the pre-cleaning apparatus comprises one or more of a nozzle and an outlet port, wherein one or more of the nozzle and the outlet port are arranged in the instrument tray.

19. The method according to claim 11, wherein the pre-cleaning apparatus comprises a fluid supply configured to hold a pre-cleaning fluid for pre-cleaning the outside of the surgical instrument.

20. The method according to claim 11, wherein the pre-cleaning apparatus comprises a fluid pump connected to the fluid supply by a fluid feed line.

21. The method according to claim 11, further comprising a fluid outlet line configured to remove pre-cleaning fluid from the instrument tray.
